(19) United States
(12) Patent Application Publication
(10) Pub. No.: US 2008/0103479 A1
(43) Pub. Date: May 1, 2008

(54) Delivery system using balloon catheter with side opening and method

(76) Inventors: Eric Cheng, Bloomington, IN (US); Susana M. Deutsch, Coral Gables, FL (US); Larry Dominguez, West Miami, FL (US); Ajay K. Wakhloo, Wellesley, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/893,667

(22) Filed: Aug. 17, 2007

Related U.S. Application Data
(63) Continuation-in-part of application No. 10/681,602, filed on Oct. 8, 2003, now abandoned.
Application No. 10/681,602 is a continuation of application No. 09/878,530, filed on Jun. 11, 2001, now abandoned.

(51) Int. Cl. A61M 25/10

(52) U.S. Cl. 604/510

(57) ABSTRACT

Methods for placing a medical agent at a location within a patient's vessel. In an illustrative embodiment, an embolic coil is placed at a location with an aneurysm. The methods include the steps of providing a catheter having a proximal end and a distal end, a balloon adjacent to the distal end, an inflation port at the proximal end communicating via an inflation lumen with the balloon, a delivery port at the proximal end communicating with a delivery lumen, a guidewire opening at the distal end communicating with the delivery lumen, and a side opening near the distal end and spaced from the guidewire opening and also communicating with the delivery lumen. The catheter is pre-loaded with a guidewire extending from the delivery port through the delivery lumen and distal of the guidewire opening. Thereafter, the catheter is introduced into the vessel of a patient to generally align the side opening with the aneurysm. The balloon is inflated to stabilize the position of a distal portion of the catheter. Thereafter, the guidewire is withdrawn and an embolic coil employment device is introduced into the delivery lumen and through the side opening to deliver an embolic coil into the aneurysm. Once the desired number of embolic coils are delivered into the aneurysm, the balloon is deflated and the catheter is withdrawn from the patient's vessel.

### REFERENCE TO RELATED APPLICATION

This patent application is a continuation-in-part of U.S. patent application Ser. No. 10/681,602 entitled “Delivery System Using Balloon Catheter”, filed Oct. 8, 2003, which is a continuation of Ser. No. 09/878,530 entitled “Delivery System Using Balloon Catheter”, filed Jun. 11, 2001.

### FIELD OF THE INVENTION

This invention concerns medical devices and methods, including delivery systems using a balloon catheter with a side opening.

### BACKGROUND AND SUMMARY

In general, treating an aneurysm by placing embolic coils inside it is well known. Various devices are available for delivering the embolic coils through the patient's vessel to the aneurysm. Typically these embolic coils, which generally take the form of helically wound coils or random wound coils, are coupled to a coil deployment device which serves to introduce the coils into the aneurysm, and then enables release of the coils through various types of release mechanisms. It is desirable to place the coils in the exact position desired, but some measure of deployment device motion within the vessel may prove challenging during the introduction of the embolic coil to the aneurysm.

It is, therefore, an object of the invention to provide a method for placing embolic coils in a relatively precise manner, with enhanced stability.

Another object of the present invention is to provide a system for placing embolic coils within an aneurysm, which system is relatively efficient and simple for the physician to operate.

A further object of the present invention is to provide a method for delivering guidewires, embolics, diagnostic, and therapeutic agents via a delivery lumen in a relatively simple, efficient and stable manner.

A still further object is to provide a delivery catheter that enables the delivery of embolic coils within an aneurysm in a relatively simple, stable and effective manner.

Another object of the present invention is to provide a delivery catheter that can be utilized to deliver guidewires, embolics, diagnostic, and therapeutic agents via a delivery lumen.

A further object of the present invention is to provide a delivery catheter that is relatively simple in construction and easy to manufacture.

Other objects of the present invention will become apparent as the description proceeds.

In accordance with the principles of the present invention, novel methods are provided for placing a medical agent at a location to be treated within the vessel of a patient. The medical agent may be one or more embolic coils, or any other suitable drug or medical device. The methods include the steps of providing a catheter having a proximal end and a distal end, a balloon adjacent to the distal end, and an inflation port at the proximal end communicating via an inflation lumen with a balloon. A guidewire opening is provided at the distal end, and a spaced side opening is provided adjacent to the distal end. The catheter is introduced into the vessel of a patient via a guidewire which extends through the guidewire opening to generally align the side opening with the location to be treated. The balloon is inflated to stabilize the position of the catheter distal segment. A medical agent is thereafter introduced from the proximal end of the catheter and through the side opening to deliver the medical agent to the location within the patient's vessel to be treated. Thereafter, the balloon is deflated, and the catheter is withdrawn from the patient's vessel.

In accordance with an illustrative embodiment of the present invention, novel methods are provided for placing an embolic coil or coils at a location within an aneurysm. The methods include the steps of providing a catheter having a proximal end and a distal end, a balloon adjacent to the distal end, an inflation port at the proximal end communicating via an inflation lumen with the balloon, a guidewire opening at the distal end, and a spaced side opening adjacent the distal end. The catheter is introduced into the vessel of a patient via a guidewire extending through the guidewire opening to generally align the side opening with the aneurysm. The balloon is inflated to stabilize the position of the catheter distal end, and an embolic coil deployment device is introduced to the proximal end of the catheter through the side opening via a delivery lumen to delivery an embolic coil into the aneurysm. Once the desired number of embolic coils are delivered into the aneurysm, the balloon is deflated and the catheter is thereafter withdrawn from the patient.

In the illustrative embodiment, a delivery port is provided at the proximal end communicating with the delivery lumen. The guidewire opening at the distal end also communicates with the delivery lumen.

In the illustrative embodiment, the catheter is preloaded with a guidewire extending from the delivery port through the delivery lumen and distal of the guidewire opening.

In the illustrative embodiment, a balloon catheter is provided. The balloon catheter comprises a catheter body having a proximal end and a distal end. A balloon is located adjacent to the catheter distal end, and an inflation port is located at the proximal end. The catheter body defines an inflation lumen, with the inflation port communicating via the inflation lumen with the balloon. A delivery port is provided at the proximal end of the catheter. The catheter body defines a delivery lumen that is separate from the inflation lumen. A guidewire opening is provided at the distal end communicating with a delivery lumen. A side opening is provided adjacent to the distal end, spaced from the guidewire opening, and communicating with a delivery lumen. The balloon is radially aligned with the side opening, and is positioned on the catheter opposite to the side opening.

In one embodiment of the invention, the guidewire opening and the side opening both communicate with the delivery lumen. In another embodiment of the invention, the guidewire opening communicates with a guidewire lumen, and the side opening communicates with a separate delivery lumen.

In accordance with one embodiment of the invention, a hydraulic deployment system is utilized for delivering an embolic coil, via a catheter, to an aneurysm. The hydraulic deployment device includes a positioning catheter having a distal tip for retaining an embolic coil. When the positioning catheter is pressurized with a fluid, the distal tip expands outwardly to release the coil at the preselected position within the aneurysm.

A more detailed explanation of the invention is provided in the following description and claims, and is illustrated in the accompanying drawings.

### BRIEF DESCRIPTION OF THE DRAWINGS

FIG. 1 is a partial sectional view of a delivery catheter constructed in accordance with the principles of the present invention;

FIG. 2 is a partial sectional view of a vascular occlusive coil deployment system that can be used with the delivery catheter of FIG. 1;

FIG. 3 is a diagrammatic view the delivery catheter of FIG. 1 in use to deliver an embolic coil to an aneurysm;

FIG. 4 is a cross-sectional view of the catheter of FIG. 3, taken along the plane of the line 4-4′ of FIG. 3;

FIG. 5 is a cross-sectional view of the catheter of FIG. 3, taken along the plane of the line 5-5′ of FIG. 3;

FIG. 6 is a cross-sectional view of the catheter of FIG. 3, taken along the plane of the line 6-6′ of FIG. 3;

FIGS. 7-10 are diagrammatic sequential views of a method of placing embolic coils in accordance with an embodiment of the present invention.

### DETAILED DESCRIPTION OF THE ILLUSTRATIVE EMBODIMENT

Referring to the drawing figures, a balloon catheter 10 is illustrated. Balloon catheter 10 includes a tubular catheter body 12 having a proximal end 14 and a distal end 16. At the proximal end 14 there is an inflation port 18 and a delivery port 20. A flexible balloon 22 is located adjacent distal end 16, and is in fluid communication with an inflation lumen 24, which is in fluid communication with inflation port 18. A secondary port (not shown) may also be carried by the catheter and may be in fluid communication with inflation lumen 24. The secondary port, and inflation lumen 24 could be used to purge air trapped in the balloon and tubular catheter body.

The catheter body and balloon are preferably formed of one or more polymers, according to catheter manufacturing methods that are known in the art. As a non-limiting example, the proximal portion of the catheter may be formed from nylon and the remainder of the catheter to its distal end may be formed of polyurethane. The inflatable balloon 22 may be formed from silicone or nylon materials, although it is understood that various other balloon materials and other materials for forming a catheter may be used as is known in the art of making balloon catheters.

Catheter body 12 also defines a delivery lumen 26 which communicates with delivery port 20. Catheter body 12 also has a side opening 28 which is oppositely and radially aligned with balloon 22 and is in communication with delivery lumen 26.

Also in communication with delivery lumen 26 is a guidewire opening 30, which is axially aligned with the catheter body 12 and is generally perpendicular to side opening 28.

FIG. 5 shows a cross-section of balloon 22 when it is fully inflated; FIG. 6 shows a cross-section of catheter 10 distal of 22.

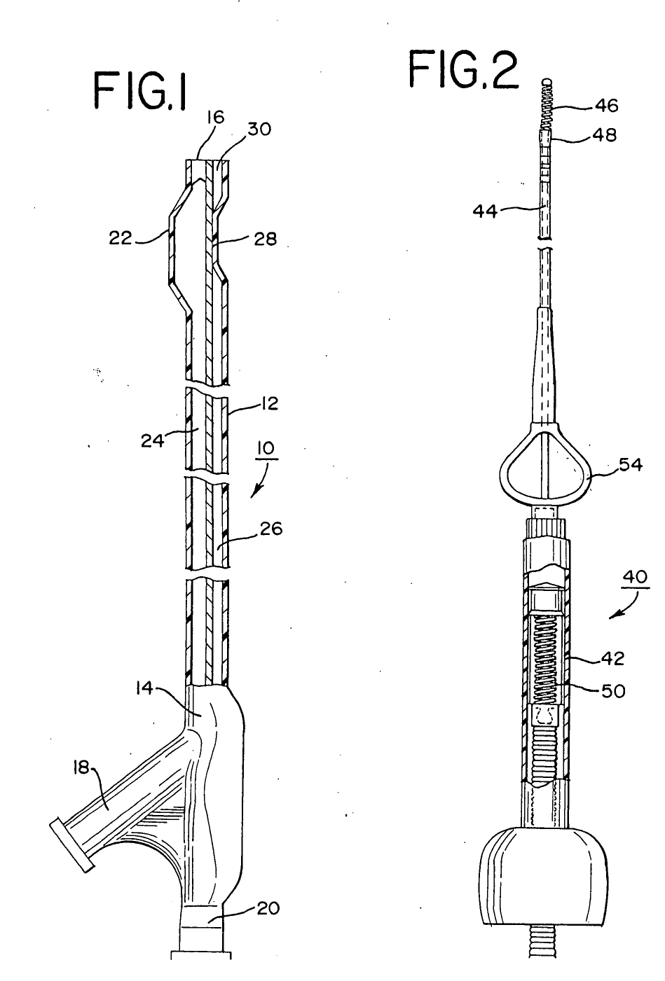

In FIG. 2, there is illustrated a deployment device 40 for embolic coils. It is understood that the device illustrated in FIG. 2 for deploying embolic coils is only one example of numerous possible deployment devices that may be used with the present invention. Reference is also made to U.S. Pat. No. 6,113,622, entitled “Embolic Coil Hydraulic Deployment System” issued Sep. 5, 2000, the disclosure of which is incorporated herein, for more details of example structure and operation of the embolic coil deployment device of FIG. 2.

Although a preferred embodiment of the invention concerns the deployment of embolic coils, it is understood that the invention can be used for the deployment of other medical agents, including liquid embolic agents, biocompatible polymer-solvent combinations, biocompatible polymers, and other embolizing compositions as are known in the art. Further, the medical agent that is deployed could be a diagnostic agent or a therapeutic agent. Although it will be apparent from the description how other medical agents may be deployed pursuant to the present invention, since an embolic coil is a preferred embodiment the description of the invention will primarily refer to deployment of embolic coils.

The deployment device 40 of FIG. 2 includes a syringe 42, coupled to the proximal end of a catheter 44. An embolic coil 46 is disposed within the lumen of the distal end 48 of the catheter. The proximal end of the coil 46 is tightly held within the lumen of the distal section 48 of catheter 44 until the deployment system is activated for release of the coil. Syringe 42 includes a threaded piston 50 which is controlled by handle 52 for infusing fluid into the interior of the catheter 44. Catheter 44 includes a winged hub 54 which aids in the insertion of the catheter into the delivery port 20 of catheter 10.

Embolic coil 46 may take various forms and configurations. Its proximal end is within distal end 48 of catheter 44, which distal end 48 is flexible to form a fluid type seal with the proximal end of coil 46. When a hydraulic pressure is applied by piston 50 to the interior of catheter 44, the distal section 48 begins to expand radially to release coil 46 from the distal end 44 and to deploy coil 46 at the desired location, for example within an aneurysm.

It is understood that various types of deployment devices may be used, including but not limited to those operating electrically, mechanically, adhesively, magnetically, etc. and coil 46 may take numerous forms as is well known in the art.

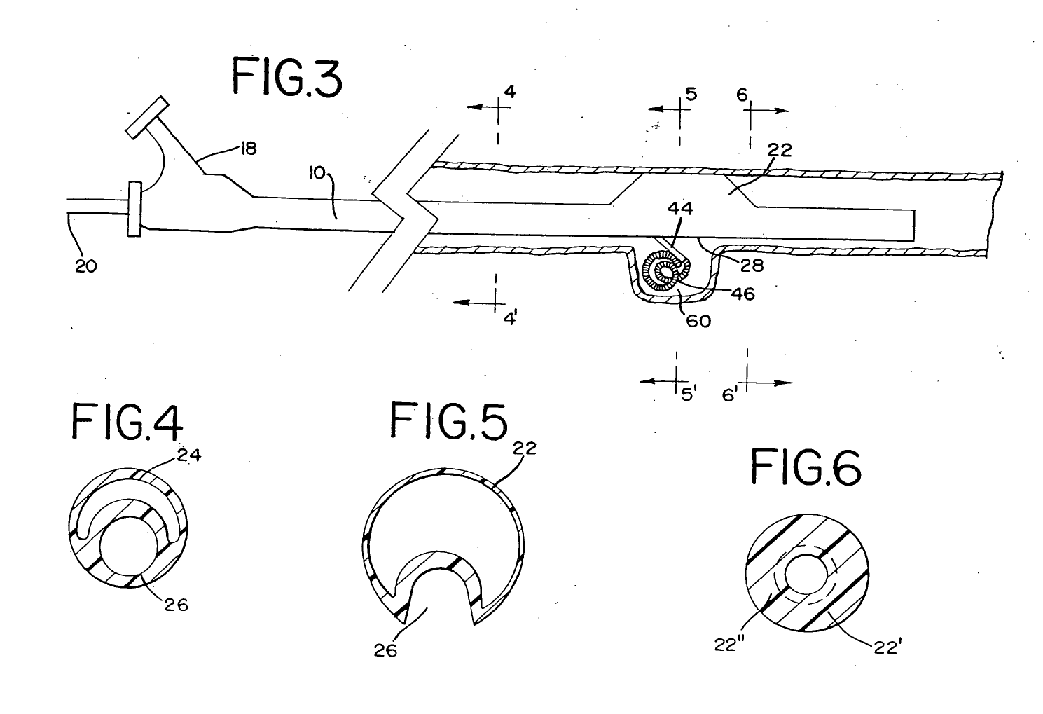

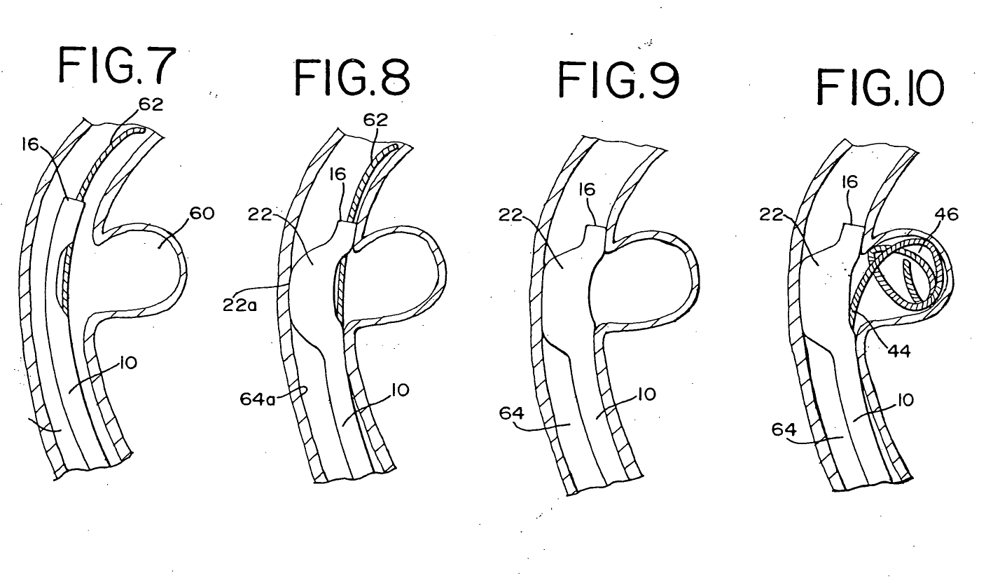

In accordance with the present invention, catheter 10 is utilized to enable a stable platform for delivery of embolic coils to an aneurysm. FIG. 3 is a diagrammatic view of catheter 10, enabling the delivery of a coil 46 to a brain aneurysm 60 through side port 28 of the catheter 10. A method for placing the embolic coil 46 at a location within aneurysm 60 is illustrated, in sequence, in FIGS. 7-10.

Referring to FIG. 7, catheter 10, which has previously been preloaded with a guidewire 62, is introduced into the patient's vessel 64. Guidewire 62 is preloaded to extend through delivery port 20, delivery lumen 26 and guidewire opening 30. The distal end of guidewire 62 is fed through a vessel, followed by catheter 10 containing guidewire 62, so that opening 28 will be aligned adjacent the aneurysm 60. Once so aligned, as illustrated in FIG. 8, balloon 22 will be inflated by providing inflation fluid via inflation lumen 24 to balloon 22 with the outer tip 22a of balloon 22 being compressed against the inner wall 64a of the vessel 64. This will serve to stabilize the catheter 10 and maintain its location within the vessel.

Once catheter 10 is in position with side opening 28 aligned with the aneurysm 60 and the balloon 22 compressed against the vessel wall, guidewire 62 is withdrawn via delivery port 20 and a catheter such as catheter 44, connected at its proximal end to a deployment device such as device 40, is inserted via delivery port 20 into delivery lumen 26. When the embolic coil 46 reaches side port 28, it will exit side port 28 into the aneurysm 60. When the coil has been placed in the desired location with the aneurysm, the coil is then released from the deployment device and the deployment device is withdrawn via the delivery port 20.

While balloon 22 remains compressed against the vessel wall, another embolic coil may be attached to the catheter 44 of the delivery device 40. The catheter 44 of the delivery device 10 is again inserted into the delivery port 20 and through the delivery lumen 26 to side port 28 so that another embolic coil will be placed in a desired location within aneurysm 60. This process can be repeated until a desired number of coils have been placed within the aneurysm. The balloon 22 is then deflated and catheter 10 is removed from the vessel.

It can be seen that a novel system has been disclosed in which a stable platform is provided for delivering a medical agent placed within an aneurysm, with a stabilized delivery/deployment catheter that is relatively simple in construction and easy to use and manufacture.

Although an illustrative embodiment of the invention has been shown and described, it is understood that various modifications and substitutions may be made by those skilled in the art without departing from the novel spirit and scope of the present invention. For example, as stated above an additional port could be used in communication with the balloon to purge air trapped in the balloon and catheter body. Instead of a single lumen used for both the guidewire and the embolic coil delivery device, a guidewire lumen which communicates with the guidewire opening at the distal end and a separate delivery lumen which communicates with the side opening could be utilized. Further, in addition to the delivery of embolics, the system can be utilized to delivery guidewires, diagnostics and therapeutic agents via a delivery lumen. The multiple lumen catheter body may be composed of polymers and/or metals, and a balloon may be pre-formed and attached to the inflation lumen adjacent the distal end of the catheter, or simply formed from the inflation lumen of the multiple lumen body. Other modifications may be made within the scope of the following claims.

What is claimed:

1. A method for placing an embolic coil at a location within an aneurysm comprising the steps of:
providing a catheter having a proximal end and a distal end, a balloon adjacent to the distal end, and an inflation port at the proximal end communicating via an inflation lumen with the balloon, a guidewire opening at the distal end, and a spaced side opening near the distal end;
introducing the catheter into the vessel of a patient via a guidewire extending through the guidewire opening to generally align the side opening with the aneurysm;
inflating the balloon to stabilize the position of a distal segment of the catheter;
introducing an embolic coil deployment device from the proximal end of the catheter and through the side opening, and delivering at least one embolic coil into the aneurysm;
deflating the balloon; and
withdrawing the catheter and deployment device from the patient's vessel.

2. A method for placing an embolic coil at a location within an aneurysm comprising the steps of:
providing a catheter having a proximal end and a distal end, a balloon adjacent to the distal end, an inflation port at the proximal end communicating via an inflation lumen with the balloon, a delivery port at the proximal end communicating with a delivery lumen, a guidewire opening at the distal end communicating with the delivery lumen, and a side opening near the distal end also communicating with the delivery lumen;
pre-loading the catheter with a guidewire extending from the delivery port through the delivery lumen and distal of the guidewire opening;
introducing the catheter into the vessel of a patient to generally align the side opening with the aneurysm;
inflating the balloon to stabilize the position of a distal segment of the catheter;
withdrawing the guidewire and introducing an embolic coil deployment device into the delivery lumen and through the side opening to deliver an embolic coil into the aneurysm;
deflating the balloon; and
withdrawing the catheter and deployment device from the patient's vessel.

3. A method for placing a medical agent at a location within a patient's vessel, comprising the steps of:
providing a catheter having a proximal end and a distal end, a balloon adjacent to the distal end, an inflation port at the proximal end communicating via an inflation lumen with the balloon, a delivery port at the proximal end communicating with a delivery lumen, a guidewire opening near the distal end communicating with the delivery lumen, and a side opening near to the distal end also communicating with the delivery lumen;
pre-loading the catheter with a guidewire extending from the delivery port through the delivery lumen and distal of the guidewire opening;
introducing the catheter into the vessel of a patient to generally align the side opening with the location to be treated;
inflating the balloon to stabilize the position of a distal portion of the catheter;
withdrawing the guidewire and introducing the medical agent into the delivery lumen and through the side opening, whereby the side opening is placed near the location to be treated;
deflating the balloon; and
thereafter withdrawing the catheter from the patient's vessel.

4. The method as defined in claim 3, in which said medical agent comprises an embolic coil.

5. The method as defined in claim 3, in which said medical agent comprises a therapeutic agent.

6. The method as defined in claim 3, in which said medical agent comprises medicament.

7. The method as defined in claim 3, in which said medical agent comprises a diagnostic agent.

8. The method as defined in claim 3, in which said medical agent comprises an embolic agent.

9. The method as defined in claim 8, in which said embolic agent is selected from the group consisting of liquid embolic agents, biocompatible polymer-solvent combinations, biocompatible polymers and other embolizing compositions.

10. The method as defined in claim 3, wherein the side opening is positioned between a proximal and distal end of the balloon.

11. The method as defined in claim 10, wherein the balloon is eccentrically arranged on the catheter.
